(19) United States
(12) Patent Application Publication
(10) Pub. No.: US 2022/0395408 A1
(43) Pub. Date: Dec. 15, 2022

(54) Apertured Nonwoven And Absorbent Articles Having The Same

(71) Applicant: The Procter & Gamble Company, Cincinnati, OH (US)

(72) Inventors: Fancheng Wang, Beijing (CN); Xiaoxin Liu, Beijing (CN); Sijia Wang, Beijing (CN); Rong Deng, Mason, OH (US); QingQing Dai, Guangzhou (CN); Meng Chen, Beijing (CN)

(21) Appl. No.: 17/345,009

(22) Filed: Jun. 11, 2021

(51) Int. Cl. A61F 13/512; A61F 13/514; A61F 13/15

(52) U.S. Cl. CPC A61F 13/512; A61F 13/5146; A61F 13/15577; A61F 2013/15934; A61F 2013/51178

(57) ABSTRACT

An apertured nonwoven substrate is disclosed. The apertured nonwoven substrate has a first area having a first caliper, a second area having a plurality of apertures, and wherein the second area has a second caliper. A ratio of the first caliper to the second caliper is at least about 1.35, as measured according to Caliper Test, and the first area has an airy index no less than about 270%, as measured according to Caliper Test.

### FIELD OF THE INVENTION

The present invention relates to apertured nonwoven, an absorbent article comprising the nonwoven, and also methods for manufacturing the same.

### BACKGROUND OF THE INVENTION

Nonwovens including synthetic fibers formed from thermoplastic resin are widely used as sheets constituting absorbent articles such as sanitary napkins, infant disposable diapers, personal care disposable diapers, and the like.

Various nonwoven webs suggested for use as a component such as backsheets, topsheets, secondary topsheets, absorbent core components and release paper wrappers, and the like. In some configurations, nonwoven webs are supplied on rolls and moved to an absorbent article manufacturing location. During the absorbent article assembly process, nonwoven webs are unwound from the rolls and supplied to an assembly line that converts the webs of material into absorbent articles. In some instances, nonwoven webs may be relatively tightly wound on the rolls, and as such, the associated high winding pressures may compress nonwoven webs, resulting in a reduced caliper. Such compressed nonwoven webs when incorporated into an absorbent article may have a thin appearance that conveys a message of reduced softness to a consumer and/or may be aesthetically unpleasing. They may also negatively affect various performances of the nonwoven webs. To mitigate the problems associated with web compression, some manufacturers may apply heat to the web materials once unwound from the rolls. In turn, the application of heat to some types of web materials may increase the caliper or volume of the web materials, referred to herein as “relofting”. Heat may be applied to the web materials in various ways.

It may be desirable that nonwovens have apertures as considered that nonwoven having apertures may have a breathable appearance, and delight users with a unique pattern especially when the apertures form a pattern in addition to enhancing fluid handling performance.

Meanwhile, one of the desirable qualities of topsheets is provision of an appropriate level of cushioning and a desirable bulkiness recovery characteristic. Another desirable quality of topsheets is the ability to reduce ponding of the fluids on topsheets before the fluids are able to be absorbed by the absorbent article. Stated another way, one design criteria of topsheets is to reduce the amount of time the fluids spend on the topsheets prior to being absorbed by the absorbent article. If the fluids remain on the surfaces of the topsheets for too long of a period of time, wearers may not feel dry and discomfort may increase. Another desirable quality of a topsheet is prevention of fluid flow-back through a topsheet and provision of dryness feel. To solve the problem of the wearer's skin feeling wet because of prolonged fluid residency on topsheets, apertured topsheets have been used to allow for faster fluid penetration into the absorbent article. Although apertured topsheets have generally reduced fluid pendency on topsheets, it may result in fluid flow-back through a topsheet. In addition, aperturing nonwoven tend to negatively impact on a caliper and a desirable bulkiness.

Meanwhile, fibers in nonwoven webs acquire and retain some fluid in small capillaries that might exist between the fibers which may be visually perceptible to the user of the product as an undesirable stain. It is also a desirable characteristic of absorbent article to present a clean user contacting surface with less stain.

There is a continuous need for apertured nonwoven having an appropriate amount of cushioning and a desirable bulkiness recovery with a lofted caliper.

There is also a continuous need for absorbent articles with reduced fluid flow-back through a topsheet.

There is also a continuous need for absorbent articles having improved surface cleanness against body fluid.

### SUMMARY OF THE INVENTION

The present invention provides a nonwoven substrate comprising a first area having a first caliper, as measured according to Caliper Test; and a second area comprising a plurality of apertures, the second area having a second caliper measured according to Caliper Test, wherein a ratio of the first caliper to the second caliper is at least about 1.35, as measured according to Caliper Test, and wherein the first area has an airy index no less than about 270%%, as measured according to Caliper Test.

The present invention also provides a relofted nonwoven substrate comprising a first area having no aperture and a second area comprising at least one aperture, the one aperture having at least three adjacent apertures, wherein an edge-to-edge space between the one aperture and each of the at least three adjacent apertures is no greater than about 3 mm.

The present invention also provides a method for manufacturing an apertured nonwoven substrate, comprising the steps of: forming a plurality of apertures on a nonwoven web, and applying energy to the nonwoven web to increase bulkiness of the nonwoven web.

The present invention also provides a method for manufacturing an apertured nonwoven substrate, comprising the steps of: applying energy to the nonwoven web to increase bulkiness of the nonwoven web; and forming a plurality of apertures on a nonwoven web.

These and other features, aspects, and advantages of the present invention will become evident to those skilled in the art from a reading of the present disclosure.

### BRIEF DESCRIPTION OF THE DRAWINGS

FIG. 1 is a plan view photo of an apertured nonwoven substrate.

FIG. 2 is a plan view photo of another apertured nonwoven substrate.

FIG. 3 is a plan view photo of another apertured nonwoven substrate.

FIG. 4 is a cross-section view photo of the apertured nonwoven substrate in FIG. 1 along A-A.

FIG. 5 is a perspective view of an absorbent article.

FIG. 6 is a schematic illustration of one exemplary process for aperturing nonwoven substrate.

FIG. 7 is a schematic side view of one exemplary relofting unit.

FIG. 8A is a perspective view of a strikethrough plate for acquisition time measurement.

FIG. 8B is a plan view of the strikethrough plate of FIG. 8A.

FIG. 8C is a plan view of a 11C-11C direction cross section of the strikethrough plate of FIG. 8B.

FIG. 8D is a plan view of part pf the strikethrough plate of FIG. 8B.

FIG. 8E is a plan view of a 11E-11E direction cross section of the strikethrough plate of FIG. 8B.

### DETAILED DESCRIPTION OF THE INVENTION

All ranges are inclusive and combinable. The number of significant digits conveys neither limitations on the indicated amounts nor on the accuracy of the measurements. All numerical amounts are understood to be modified by the word “about” unless otherwise specifically indicated.

The term “absorbent articles”, as used herein, include disposable diapers, sanitary napkins, panty liners, incontinence pads, interlabial pads, breast-milk pads, sweat sheets, animal-use excreta handling articles, animal-use diapers, and the like.

The term “component” of an absorbent article, as used herein, refers to an individual constituent of an absorbent article, such as a topsheet, secondary layer, acquisition layer, liquid handling layer, absorbent core or layers of absorbent cores, and backsheets.

### Nonwoven Substrate

The present invention provides a nonwoven substrate suitable for a component of an absorbent article. A nonwoven substrate according to the present invention comprises a first area having a first caliper, and a second area comprising a plurality of apertures and having a second caliper.

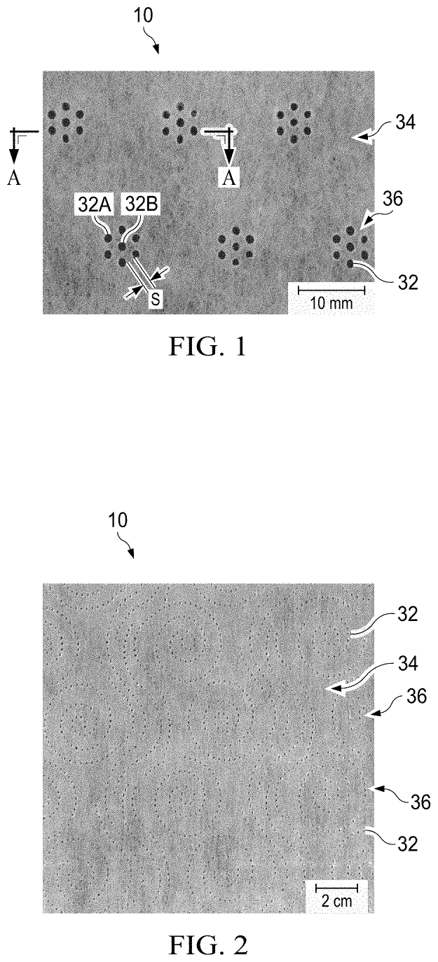

Referring to FIGS. 1 and 2 which are photo images of exemplary nonwoven substrates according to the present invention, a nonwoven substrate 10 comprises a first area 34, and a second area 36 comprising a plurality of apertures 32. Referring to FIG. 1 and FIG. 4 which is a cross-section view of nonwoven of FIG. 1 along a line A-A, the second area 36 comprises a plurality of apertures where fibers in peripheries of apertures are compressed and heat-fused in comparison with fibers in the first area.

While the first area 34 and the second area 36 will be discussed individually in greater details in dedicated sections further below, this section will briefly discuss the nonwoven substrate 10 comprising these two areas, as a whole.

The first area has a first caliper, and the second area comprising a plurality of apertures has a second caliper. The nonwoven substrate has a ratio of the first caliper to the second caliper at least about 1.35, or at least about 1.4, or at least about 1.5, or at least about 1.6, as measured according to Caliper Test disclosed herein.

In some embodiments, the nonwoven substrate of the present invention may comprise a plurality of first areas. The first areas (i.e., non-apertured portions) may be in a regular, homogeneous and uniform shape, or an irregular and non-uniform shape. At least some of the first area in the nonwoven substrate surround at least some of the plurality of second areas.

The nonwoven substrate of the present invention may meet certain parametric requirements as detailed below. Among the parameters of interest, Compression Work (CW) is obtained from the FTT Test under Compression Property Test below. Information about the FTT Test method may be found in the paper “Fibers and Polymers 2014, Vol. 15, No. 7, 1548-1559” titled “A Simultaneous Measurement Method to Characterize Touch Properties of Textile Materials” by Xiao Liao et al.

The nonwoven substrate may have a compression work no less than about 700 gf×mm, or no less than about 800 gf×mm, or no less than about 900 gf×mm as measured according to Compression Properties described under the TEST METHODS. Compression work is a parameter which can indicate compliance property of a specimen by quantifying the total work done on the specimen during the compression process. A higher compression work in nonwoven may represent a more cushiony property which drives desired consumer benefits of a softness perception as well as comfortable usage experience. Without wishing to be bound by theory, it is believed that the nonwoven substrate of the present invention may exhibit high compression work due to a lofted and airy structure in the first area.

The apertured nonwoven substrate of the present invention may be a relofted nonwoven. Relofting process is a process which provide heat or energy a nonwoven web so that the nonwoven web regain its bulkiness.

The nonwoven substrate of the present invention may comprise one or more layers.

In some embodiments, the nonwoven substrate comprises at least two layers each of which remains as a discrete layer which may be attached to each other by, for example, thermal bonding, compression, adhesive bonding or any combination thereof. The first layer and the second layer in the nonwoven substrate may be bonded to each other without using chemicals such as adhesive and latex.

In some embodiments, the nonwoven substrate comprises a unitary structure. A unitary structure herein intends to mean that although it may be formed by several sub-layers that have distinct properties and/or compositions from one another, they are somehow intermixed at the boundary region, so that, instead of a definite boundary between sub-layers, it would be possible to identify a region where the different sub-layers transition one into the other. Such a unitary structure is typically built by forming the various sub-layers one on top of the other in a continuous manner, for example using air laid or wet laid deposition. Typically, there is no adhesive used between the sub-layers of the unitary material. However, in some cases, adhesives and/or binders can be present although typically in a lower amount that in multilayer materials formed by separate layers.

The nonwoven substrate may comprise thermoplastic fibers. The nonwoven substrate may comprise any suitable types of thermoplastic fibers, such as polypropylene fibers, other polyolefins, other polyesters besides PET such as polylactic acid, thermoplastic starch-containing sustainable resins, other sustainable resins, bio-PE, bio-PP, and Bio-PET. The nonwoven substrate may comprise any other suitable types of fibers such as viscose fibers, rayon fibers, or other suitable nonwoven fibers, for example. These fibers may have any suitable deniers or denier ranges and/or fiber lengths or fiber length ranges.

The nonwoven substrate may comprise bicomponent fibers. Bicomponent fibers can have a sheath and a core. The sheath and the core may also comprise any other suitable materials known to those of skill in the art. The core/sheath composite fibers may comprise a core component comprising a resin and a sheath component comprising a thermoplastic resin having a melting point of at least about 20° C. lower than a melting point of the resin of the core component. The sheath and the core may each comprise about 50% of the fibers by weight of the fibers, although other variations (e.g., sheath 60%, core 40%; sheath 30%, core 70% etc.) are also within the scope of the present disclosure. The bicomponent fibers or other fibers that make up the first and/or second layers may have a denier in the range of about 0.5 to about 6, about 0.75 to about 4, about 1.0 to about 4, about 1.5 to about 4, about 1.5 to about 3, about 1.5 to about 2.5, or about 2, specifically including all 0.1 denier increments within the specified ranges and all ranges formed therein or thereby. Denier is defined as the mass in grams per 9000 meters of a fiber length. In other instances, the denier of the fibers of the first layer may be in the range of about 1.5 denier to about 6 denier or about 2 denier to about 4 denier and the denier of the fibers of the second layer may be in the range of about 1.2 denier to about 3 denier or about 1.5 denier to about 3 denier, specifically reciting all 0.1 denier increments within the specified ranges and all ranges formed therein or thereby. Bicomponent fibers can be a side-by-side type fibers.

In a form, the basis weight of the nonwoven substrate may be appropriately selected depending on the nonwoven application. For the nonwoven of the present invention as a topsheet of an absorbent article, a basis weight of the nonwoven substrate may be from about 15 gsm (g/m²) to about 75 gsm, or from about 20 gsm to about 75 gsm, or from about 30 gsm to about 65 gsm. All other suitable basis weight ranges for the nonwoven substrate are within the scope of the present disclosure. Accordingly, the basis weight of the nonwoven substrate may be designed for specific product requirements.

The nonwoven substrate may further comprise three-dimensional elements such as protrusions and/or recesses on a wearer-facing surface of the nonwoven substrate. The nonwoven substrate comprises protrusions, the protrusion may have a higher caliper than the first caliper in the first area.

As used herein, the term “nonwoven” or “nonwoven substrate” refers to a web having a structure of individual fibers or threads which are interlaid, but not in a repeating pattern as in a woven or knitted fabric, which do not typically have randomly oriented fibers. Nonwoven substrates or fabrics have been formed from many processes, such as, for example, meltblowing, spunbonding, hydroentangling, airlaid, wetlaid, through-air-dried paper making processes, and bonded carded web processes, including carded thermal bonding. The nonwoven substrates can comprise unbonded fibers, entangled fibers, tow fibers, or the like. Fibers can be extensible and/or elastic, and may be pre-stretched for processing. Fibers can be continuous, such as those produced by spunbonded methods, or cut to length, such as those typically utilized in a carded process. Fibers can be bicomponent, multiconstituent, shaped, crimped, or in any other formulation or configuration known in the art for nonwoven substrates and fibers. In general, the fibers can be bondable, either by chemical bond (e.g. by latex or adhesive bonding), pressure bonding, or thermal bonding. If thermal bonding techniques are used in the bonding process described below, a certain percentage of thermoplastic material, such as thermoplastic powder or fibers can be used.

The nonwoven of the present invention has an appropriate amount of cushioning and bulkiness recovery characteristics.

As such, the nonwoven of the present invention can be preferably used in applications in which the nonwoven is in contact with the skin, specifically applications in which the first web layer is the surface that is in contact with the skin. The nonwoven of the present invention is preferably used as a topsheet for an absorbent article in which the surface of first web layer is in contact with the skin.

First Area

Referring to FIG. 1, nonwoven substrate 10 of the present invention comprises a first area 34. The first area 34 is a land area, i.e., non-apertured area, having a first caliper.

The first area may have an airy index, no less than about 270%%, or no less than about 300%, or no less than about 350%, as measured according to Caliper Test. The first area may have a recovery airy index no less than about 160%, or no less than about 180%, or no less than about 200%, no less than about 220%, as measured according to Caliper Test. High airy index relates to an airy perception, both airy and recovery airy index link to a light touch cushiony feel.

Second Area

Referring to FIG. 1, nonwoven substrate 10 of the present invention comprises a second area 36 comprising plurality of apertures 32. Second area 36 has a second caliper measured according to Caliper Test disclosed herein. The second caliper is smaller than the first caliper of the first area.

Apertures may be in any of circular, oval, hour-glass shaped, star shaped, polygonal and the like, and combinations thereof. Polygonal shapes include, but are not limited to triangular, quadrilateral, hexagonal, octagonal or trapezoidal. In one embodiment, apertures are circular. In another embodiment, apertures are an oval shape. Apertures may have a size in a range of about 0.1 mm²-about 3 mm², or in a range of about 0.2 mm²-about 2 mm², or in a range of about 0.3 mm²-about 1 mm². The second area may have apertures having the same size and/or shape. The second area may have apertures having different sizes and/or shapes.

The second area may comprise apertures forming a pattern. A pattern formed by apertures may be any shape of pattern, for example, a shape of one or multiple linear lines or curved lines, a circles, an ellipse, a triangle, a polygon, a flower, a cloud, and the like. The pattern may be a regular, homogeneous and uniform pattern or an irregular, non-uniform and non-homogeneous pattern. In some embodiments, a nonwoven substrate of the present invention comprises a plurality of second areas, wherein apertured patterns in the second areas are not necessarily in the same shape or size. That is, an apertured pattern in one second area may differ from an apertured pattern in another second area in the nonwoven substrate of the present invention. Patterns may be various shapes and/or various sizes. The nonwoven substrate of the present invention may have uniform aperture patterns.

In some embodiments, the nonwoven substrates comprising a second area comprising clustered apertures. The term “clustered apertures” herein intends to mean an aperture pattern wherein at least one aperture having at least three adjacent apertures, wherein the one aperture and each of the at least three adjacent apertures has an edge-to-edge space (shortest space between an edge of one aperture to an edge of an adjacent aperture) no greater than about 3 mm Referring to FIG. 1, aperture 32A has three adjacent apertures, and aperture 32B has six adjacent apertures.

In addition, such a nonwoven structure that has the second area having more compressed and heat-fused fibers than the first area also helps create local capillarity gradient. Referring to FIGS. 1 and 4, the fluid travels from the first area 34 having a low capillarity to the second area 36 having clustered apertures and a high capillarity due to the capillarity difference. As a result, the nonwoven substrate of the present invention can have a lower rewet amount and a smaller stain area when used as a topsheet of an absorbent article.

The aperture pattern in a second area may coordinate with graphics, indicia, printing, inks, color, and/or patterned adhesives, for example, located in the nonwoven substrate or in another component of the absorbent article when it is used as a component of an absorbent article.

### Absorbent Article

The present invention is directed to an absorbent article comprising a topsheet, a backsheet, an absorbent structure disposed between the topsheet and the backsheet, and the apertured nonwoven substrate of the present invention. In some embodiments, the absorbent article comprises a topsheet comprising the apertured nonwoven substrate of the preceding invention.

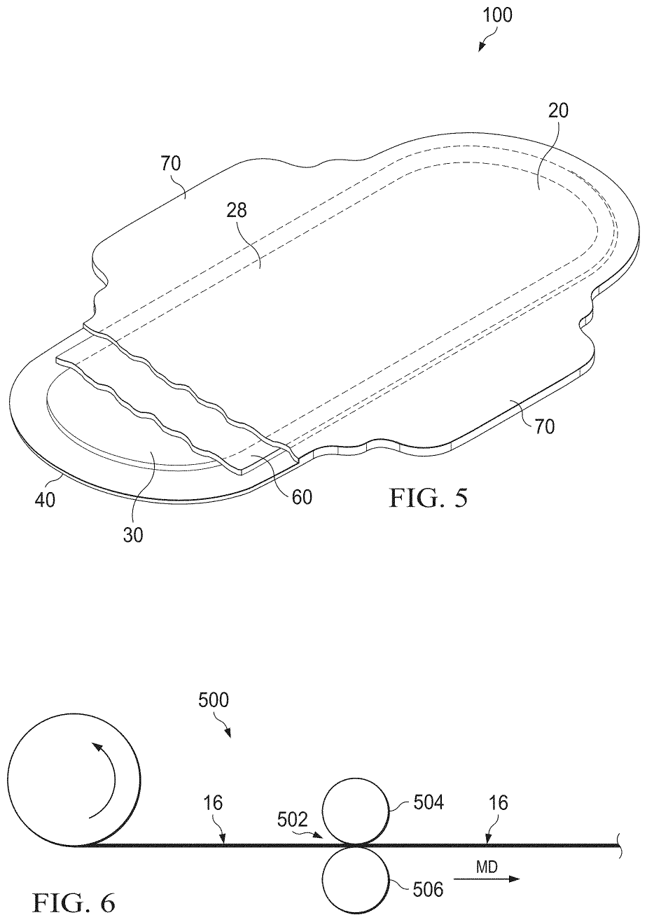

For the purposes of a specific illustration, FIG. 5 shows an example of an absorbent article 100 that may be include components made from a nonwoven substrate disclosed herein. In particular, FIG. 5 shows one example of a plan view of an absorbent article 100 configured as a sanitary napkin.

As shown in FIG. 5, an absorbent article 100 according to the present invention comprises a topsheet 20 having a body facing surface 28 and a garment facing surface (not indicated in FIG. 5) positioned opposite to the body facing surface 28. The absorbent article 100 further comprises a backsheet 40 having a garment facing surface and a user facing surface positioned oppositely to the garment facing surface, the backsheet 40 being at least partially joined to the topsheet 20. The absorbent article 100 also comprises an absorbent core 30 positioned between the topsheet 20 and the backsheet 40. The absorbent article 100 may further comprise a secondary topsheet 60 and/or a pair of flaps or wings 70. The topsheet 20, the backsheet 40, and the absorbent core 30 can be assembled in a variety of well-known configurations.

The backsheet 40 and the topsheet 20 can be secured together in a variety of ways. The topsheet 20 and the backsheet 40 can be joined to each other by using an adhesive, heat bonding, pressure bonding, ultrasonic bonding, dynamic mechanical bonding, or a crimp seal. A fluid impermeable crimp seal can resist lateral migration (“wicking”) of fluid through the edges of the product, inhibiting side soiling of the user's undergarments.

When the absorbent article is a sanitary napkin as shown in FIG. 5, as is typical for sanitary napkins and the like, the sanitary napkin can have panty-fastening adhesive disposed on the garment facing side of backsheet 40. The panty-fastening adhesive can be any of known adhesives used in the art for this purpose, and can be covered prior to use by a release paper, as is well known in the art. If flaps or wings are present, panty fastening adhesive can be applied to the garment facing side so as to contact and adhere to the underside of the user's panties.

Topsheet

In the present application, a topsheet is the part of an absorbent article that is in contact with the user's skin. The topsheet may be joined to a backsheet, an absorbent core and/or any other layers as is known to those of skill in the art. Usually, the topsheet and the backsheet are joined directly to each other in some locations (e.g., on or close to the periphery of the absorbent article) and are indirectly joined together in other locations by directly joining them to one or more other components of the article.

The topsheet may be compliant, soft-feeling, and non-irritating to the user's skin. Further, a portion of, or all of, the topsheet may be liquid permeable, permitting liquids to readily penetrate through its caliper.

The topsheet comprises a nonwoven layer comprising a nonwoven substrate of the present disclosure. The topsheet can be a composite or a laminate comprising a nonwoven layer comprising a nonwoven substrate of the present disclosure. In any of various configurations, the nonwoven substrate of the present disclosure is intended to form at least a portion of the body facing surface of an absorbent article in such a way that apertures are towards in an absorbent core of the absorbent article.

The topsheet can also optionally include colorants, such as pigment, lake, toner, dye, ink or other agent used to impart a color to a material. Suitable pigments herein include inorganic pigments, pearlescent pigments, interference pigments, and the like.

Any portion of the topsheet may be coated with a lotion and/or a skin care composition as is generally disclosed in the art.

The topsheet comprises a plurality of apertures to ease penetration of fluids and/or air therethrough. The size of at least the primary apertures may be determined to achieve the desired fluid and/or air penetration performance and other performances expected by wearers. If the apertures are too small, the fluids may not pass through the apertures, either due to poor alignment of the fluid source and the aperture location or due to runny fecal masses, for example, having a diameter greater than the apertures. If the apertures are too large, the area of skin that may be contaminated by “rewet” from the article is increased.

The topsheet may comprise a plurality of embossments to provide a more cloth like appearance.

The topsheet may be formed of any basis weight. However, relatively higher basis weight, while having relatively greater apparent caliper and loft, also has relatively greater cost. Suitable basis weight for the topsheet may be 200 gsm or less, or from 15 gsm to 80 gsm, or from 20 gsm to 70 gsm, or from 15 gsm to 60 gsm.

Absorbent Core

An absorbent core of an absorbent article serves to store bodily fluids discharged during use. The absorbent core can be manufactured in a wide variety of sizes and shapes, and may be profiled to have different caliper, hydrophilic gradients, superabsorbent gradients, densities, or average basis weights at different positions across the face of the product.

An absorbent core may comprise a fluid distribution layer as well as a fluid storage layer. The fluid distribution layer transfers received fluid both downwardly and laterally, and generally has more permeability and less capillarity than the fluid storage layer.

In addition to conventional absorbent materials such as creped cellulose wadding, fluffed cellulose fibers, wood pulp fibers also known as airfelt, and textile fibers, the fluid storage layer often includes superabsorbent material that imbibe fluids and form hydrogels. These materials are typically capable of absorbing large quantities of body fluids and retaining them under moderate pressures. The fluid storage layer of the absorbent core may comprise a superabsorbent material dispersed in a suitable carrier such as cellulose fibers in the form of fluff or stiffened fibers. The fluid storage layer of the absorbent core may comprise a superabsorbent material and be free from free cellulose fibers in the form of fluff or stiffened fibers.

Backsheet

The backsheet that covers the lower side of the absorbent core prevents the fluids in the absorbent core from wetting articles that contact the sanitary napkin, such as undergarments. Accordingly, the backsheet can be made from a liquid impervious thin film or a liquid impervious but vapor pervious film/nonwoven laminate, a microporous film, an apertured formed film, or other polymer film that is vapor permeable, or rendered to be vapor permeable, but substantially impervious to fluid.

Apertured Nonwoven substrate Manufacturing Process

An apertured nonwoven substrate of the present invention may be manufactured by a method comprising the steps of forming a plurality of apertures on a nonwoven web, and applying energy to the nonwoven web to increase bulkiness of the nonwoven web. In one embodiment, the method for producing an apertured nonwoven substrate of the present invention comprises the steps of: supplying a nonwoven substrate unwound from a nonwoven roll, forming a plurality of apertures on the nonwoven substrate, and then applying heat to the nonwoven to restore bulkiness of the nonwoven.

An apertured nonwoven substrate of the present invention may be manufactured via a process comprising the steps of: supplying a nonwoven substrate unwound from a nonwoven roll, applying heat to the nonwoven to restore bulkiness of the nonwoven, and forming a plurality of apertures on the nonwoven substrate.

Aperture forming process may be conducted via various processes known to those skilled in the art such as aperturing process such as pin-hole aperturing, punch aperturing, and water-jet aperturing.

FIG. 6 is a schematic illustration of mechanical aperturing process as an exemplary aperture forming process. Referring to FIG. 6, a nonwoven web 16 is passed through a nip 502 formed by a pair of rolls 500, two intermeshing rolls 504 and 506, to form an apertured nonwoven web 16. At least one of the rolls 504 and 506 may be heated to a temperature higher than the melting point of fibers constituting the nonwoven web. When the fiber comprises a sheath/core type bicopolymer, at least one of the rolls 504 and 506 may be heated to a temperature higher than the melting point of the sheath polymer.

In one embodiment, a first roll 504 may create apertures 32 in the nonwoven web 16 (in combination with the second roll). The first roll 504 may comprise a plurality of protrusions extending outwardly from the first roll 504. The protrusions on the first roll 504 may be various in a size, shape, height, area, width and/or dimension which may determine the size, shape and dimension of apertures 32. The second roll 506 may have a flat surface. Or, the second roll 506 may comprise grooves intermeshing with the protrusions of the first roll 504.

In another embodiment, a first roll 504 may create the apertures 32 (in combination with the second roll) and a second roll 506 may create projections (in combination with the first roll) in the nonwoven web 16. The first roll 504 may comprise a plurality of conically-shaped protrusions extending radially outwardly from the first roll 504. The first roll 504 may also comprise a plurality of recesses formed in a radial outer surface of the first roll 504. The second roll 506 may comprise a plurality of dome-shaped protrusions extending radially outwardly from the second roll 506. The second roll 506 may also comprise a plurality of recesses formed in the radial outer surface of the second roll 506. The protrusions on the first roll 504 may have a different size, shape, height, area, width and/or dimension than the protrusions on the second roll 506.

The apertured nonwoven of the present invention may be a relofted nonwoven. Relofting process is a process to make a nonwoven web regain its bulkiness by providing energy to the nonwoven web. Relofting process may be conducted via various processes known to those skilled in the art. A heating source includes oven, burner, or infrared radiation, producing heat to increase the temperature of the nonwoven substrate. As the temperature increases, fibers within the nonwoven substrate begin to soften, and at least some of the fibers begin to realign with, and/or detach from, the fibers. The realigning and/or detaching fibers cause the nonwoven substrate to increase in caliper, thereby decreasing the density of the nonwoven substrate. The final relofted caliper is dependent upon the temperature and the residence time, which is the overall time that the nonwoven substrate is exposed to the increased temperature in the relofting process.

In one embodiment, relofting a nonwoven can be conducted in accordance with methods disclosed in PCT/US2019/066455 filed on Sep. 5, 2019. The PCT application is hereby incorporated herein by reference in its entirety unless expressly excluded or otherwise limited.

The method comprising: advancing a nonwoven substrate in a machine direction MD, the nonwoven substrate comprising a first surface and an opposing second surface and defining a width in a cross direction; providing a first infrared radiation source; directing a first length of the nonwoven substrate to advance in a first direction such that the first surface of the first length of the nonwoven substrate is in a facing relationship with the first radiation source; and irradiating the first surface of the first length of the nonwoven substrate with infrared radiation from the first infrared radiation source, wherein the nonwoven substrate comprises a first caliper upstream of the first radiation source and wherein the nonwoven substrate comprises a second caliper downstream of the first radiation source, wherein the second caliper is higher than the first caliper.

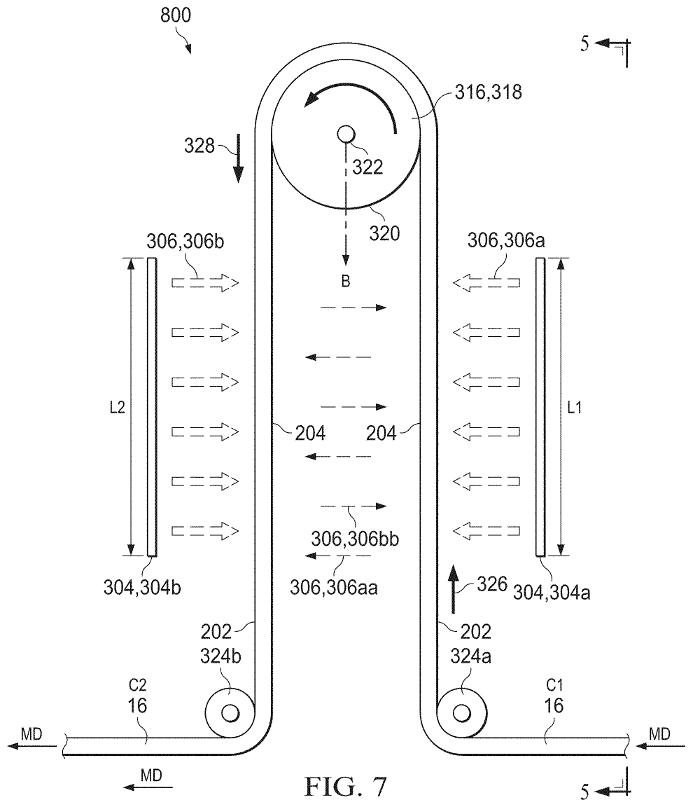

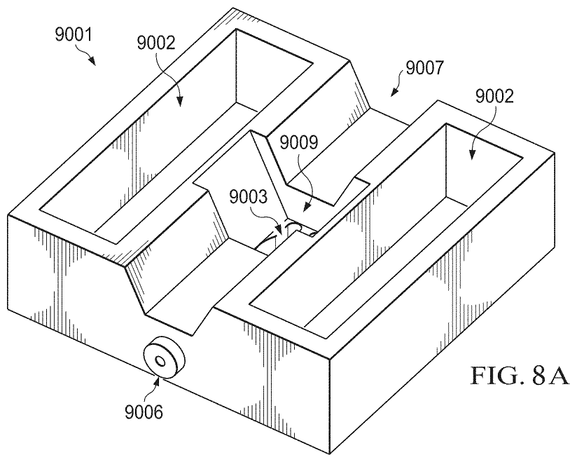

Referring to FIG. 7, the relofting unit 800 may include a first radiation source 304a and a second radiation source 304b, and a movable axis 316 that may be configured as a traversing idler 318 including an outer circumferential surface 320 adapted to rotate about an axis of rotation 322. The first movable axis 316 may be movable between a first position and a second position to place a nonwoven substrate 16 in facing relationship with (or isolate the nonwoven substrate 16 from) the first and second radiation sources 304a, 304b. In operation, a first guide roller 324a may direct the nonwoven substrate 16 in a first direction 326 to the traversing idler 318 so that a first length L1 of the first surface 202 of the nonwoven substrate 8 nonwoven substrate 16 is in a facing relationship with the first radiation source 304a, and a second length L2 of the first surface 202 of the nonwoven substrate 16 is in a facing relationship with the second radiation source 304b. The first length L1 of the nonwoven substrate 16 may advance in the first direction 326 past the first radiation source 304a, and the second length L2 of the nonwoven substrate 16 may advance in the second direction 328 past the second radiation source 304b. As such, the first radiation source 304a irradiates the first length L1 of the first surface 202 of the advancing nonwoven substrate 16 with infrared radiation 306a. And the second radiation source 304b irradiates the second length L2 of the first surface 202 of the advancing nonwoven substrate 16 with infrared radiation 306b. In turn, infrared radiation 306 heats the advancing nonwoven substrate 16 when the movable axis 316 is in the second position. As shown in FIG. 7, the nonwoven substrate 16 upstream of the relofting unit 800 comprises a first caliper C1, and the infrared radiation 306 heats the substrate such that the caliper of nonwoven substrate 16 downstream increases to a second caliper C2 that is greater than the first caliper C1.

In some configurations, some infrared radiation 306 may travel through the nonwoven substrate 16, and such infrared radiation 306 may be utilized to irradiate other portions of the nonwoven substrate 16. For example, as shown in FIG. 7 the movable axis 316 may redirect the second length L2 of the nonwoven substrate 16 from the first direction 326 to advance in the second direction 328 such that the second surface 204 of the first length L1 of nonwoven substrate 16 is in a facing relationship with the second surface 204 of the second length L2 of the nonwoven substrate 16. In turn, a portion 306aa of the infrared radiation 306a from the first radiation source 304a may travel through the first length L1 of the nonwoven substrate 16 and away from the second surface 204 of the first length L1 of the nonwoven substrate 16 and may irradiate the second surface 204 of the second length L2 of the nonwoven substrate 16. In addition, a portion 306bb of the infrared radiation 306b from the second radiation source 304b may travel through the second length L2 of the nonwoven substrate 16 and away from the second surface 204 of the second length L2 of the nonwoven substrate 16 and may irradiate the second surface 204 of the first length L1 of the nonwoven substrate 16.

It is to be appreciated that the relofting unit 800 herein may be configured to heat the advancing nonwoven substrate to various temperatures and may reloft the nonwoven substrate to increase the caliper of the substrate by various amounts. For example, in some configurations, the relofting unit 800 may heat the nonwoven substrate to temperatures ranging from about 70° C. to about 110° C., specifically reciting all 0.1° C. increments within the above-recited range and all ranges formed therein or thereby.

Aperture forming process and relofting process can be carious out consciously, or discontinuously.

### Test Methods

1. Caliper Test

Thermomechanical Analysis (TMA), Module TMA/SDTA 1 IC/600, from Mettler-Toledo AG (Switzerland) or equivalent is used to measure the local caliper changes of nonwoven materials at ambient air condition as a function of the applied force. The measurements are conducted under the compression mode, using a quartz glass sample holder and a quartz glass measuring probe with flat circular end at the size of 3 mm in diameter.

If a nonwoven is available in its raw material form, a specimen with the dimensions of about 8 mm×5 mm is cut from the raw material. If a nonwoven is a component layer such as a topsheet of an absorbent article, a nonwoven specimen of this size is removed from the absorbent article, using a razor blade to excise the component layer from the underling layers of the absorbent article. A cryogenic spray (such as Cyto-Freeze, Control Company, Houston Tex.) or other suitable solvents that do not permanently alter the properties of the nonwoven specimen composition may be used to remove the topsheet specimen from the underling layers if necessary. For apertured nonwovens, specimens are sampled from different zones of the material, allowing the measurements on an apertured zone and a non-apertured zone separately.

Any remaining adhesive may be removed from the specimen by the following steps using Tetrahydrofuran (THF) as solvent.
1) In a hood, transfer 1 liter of THF into the 3-4 liter beaker.
2) Submerge specimen in the 1 liter of THF.
3) Place beaker on shaking table and stir gently for 15 minutes and keep solution with sample sit for 5 additional minutes.
4) Take specimen out of THF solution, and carefully squeeze THF solution out of specimen.
5) Let specimen air dry in hood for a minimum of 15 minutes.

Place a specimen flat at the sample holder with the consumer facing side exposed. Adjust the specimen position so that the area of interest is located at the center of the sample holder and to be measured by the probe. The measuring probe moves down to the specimen and applies the forces in a normal direction following a method of stepwise compression (segments 1-10) and then stepwise recovery (segments 11-19) as specified below. The local caliper data of the specimen is collected in TMA at the same time with a frequency of 5 readings per second at regular intervals.

| Segment | 1 | 2 | 3 | 4 | 5 | 6 | 7 | 8 | 9 | 10 |
|---|---|---|---|---|---|---|---|---|---|---|
| Force (N) | 0.003 | 0.005 | 0.01 | 0.02 | 0.03 | 0.04 | 0.05 | 0.06 | 0.07 | 0.08 |
| Time (s) | 6 | 6 | 6 | 6 | 6 | 6 | 6 | 6 | 6 | 6 |

| Segment | 11 | 12 | 13 | 14 | 15 | 16 | 17 | 18 | 19 |
|---|---|---|---|---|---|---|---|---|---|
| Force (N) | 0.07 | 0.06 | 0.05 | 0.04 | 0.03 | 0.02 | 0.01 | 0.005 | 0.003 |
| Time (s) | 6 | 6 | 6 | 6 | 6 | 6 | 6 | 6 | 6 |

The specimen caliper measured at the midpoint (i.e. the time point of 3 s) of Segment 2 under the applied force of 0.005N or pressure of 0.1 psi is recorded as caliper at 0.1 psi. The specimen caliper measured at the midpoint of Segment 10 under the applied force of 0.08N or pressure of 1.6 psi is recorded as caliper at 1.6 psi. The specimen caliper measured during recovery at the midpoint of Segment 18 under the applied force of 0.005N or pressure of 0.1 psi is recorded as recovery caliper at 0.1 psi. Prepare and analyze a total of three substantially similar replicate samples for apertured and non-apertured zones respectively. The reported value is the arithmetic mean of the three replicate samples to the nearest integer in the unit of μm.

Caliper ratio at 0.1 psi is a caliper ratio between the non-apertured zone and the apertured zone measured at the pressure condition of 0.1 psi during compression. Airy Index and Recovery Airy Index are calculated using equations below.

Airy Index(%)=[(caliper at 0.1 psi−caliper at 1.6 psi)/caliper at 1.6 psi]×100

Recovery Airy Index(%)=[(recovery caliper at 0.1 psi−caliper at 1.6 psi)/caliper at 1.6 psi]×100

2. Compression Property Test

(1) Specimen Preparation

To obtain a nonwoven raw material specimen, lay the material flat on a bench with the technical face-side upward, and a 110 mm×110 mm square shape of specimen are cut using scissors. The technical face-side is the surface intended to be used as the body facing surface when the nonwoven is used as a component in an absorbent article.

To obtain a specimen of a topsheet nonwoven from an absorbent article, a 110 mm×110 mm topsheet specimen is removed from the absorbent article, centered at the intersection of the longitudinal and lateral centerlines of the absorbent article. For the purpose of removing the topsheet from the absorbent article, a razor blade is used to excise the topsheet from the underling layers of the absorbent article around the outer perimeter for the area of 110 mm×110 mm A cryogenic spray (such as Cyto-Freeze, Control Company, Houston Tex.) may be used to remove the topsheet specimen from the underling layers if necessary. The specimen needs to be conditioned for at least 4 hours in a room maintained at 23±2° C. and 50±5% relative humidity before testing.

(2) Compression Work

Compression work denotes the total work done on the specimen during the compression process. Integral of the compression curve according to equation (1) is obtained wherein Dₐ is the initial caliper at zero pressure, D_{c} is minimum caliper at maximum pressure, F is the measured force and D is the measured caliper during compression.

$$C W = {\int }_{{D}_{a}}^{{D}_{c}} FdD \qquad ( 1 )$$

Compression work of specimens are measured using the Fabric Touch Tester (FTT M293) running FTT system software (available from SDL Atlas), or equivalent. FTT includes five modules, which may be activated at the same time and recorded of the dynamic responses from the specimens, depending on the specimen. They include compression, thermal, bending, friction, and surface modules. The instrument is calibrated according to the manufacturer's instructions using the standard calibration fabric provided by the vendor. All testing is performed in a room maintained at 23±2° C. and 50±5% relative humidity. The test procedures are conducted according to the Operating Instructions for the FTT M293 manual.

The 110 mm×110 mm specimen with technical face-side upward is placed centrally on the lower plate. The Compression test is initiated with single surface testing mode and the specimen would be pushed downwards by the upper plate applying a continuously increasing normal force from 0-8470 gf (i.e. 0-70 gf/cm²).

Five specimens are measured, and compression work, or any subset thereof, are calculated and reported with the average value.

3. Artificial Menstrual Fluid (“AMF”) Preparation

AMF is composed of a mixture of defibrinated sheep blood, a phosphate buffered saline solution and a mucous component, and has a viscosity between 7.15 cSt to 8.65 cSt at 23±1° C.

Viscosity on the AMF is performed using a low viscosity rotary viscometer such as Cannon LV-2020 Rotary Viscometer with UL adapter (Cannon Instrument Co., State College, US) or equivalent. The appropriate size spindle for the viscosity range is selected, and instrument is operated and calibrated as per the manufacturer. Measurements are taken at 23±1 C.° and at 60 rpm. Results are reported to the nearest 0.01 cSt.

Defibrinated Sheep Blood

Defibrinated sheep blood with a packed cell volume of 38% or greater collected under sterile conditions (available from Cleveland Scientific, Inc., Bath, OH, US) or equivalent is used.

Phosphate Buffered Saline Solution

The phosphate buffered saline solution consists of two individually prepared solutions (Solution A and Solution B). To prepare 1L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1L of Solution B, add 1.42±0.005 g of sodium phosphate dibasic anhydrous and 8.50±0.005 g of sodium chloride to a 1000 mL volumetric flask and add distilled water to volume. Mix thoroughly. Add 450±10 mL of Solution B to a 1000 mL beaker and stir at low speed on a stir plate. Insert a calibrated pH probe (accurate to 0.1) into the beaker of Solution B and add enough Solution A, while stirring, to bring the pH to 7.2±0.1.

Mucous Component

The mucous component is a mixture of the phosphate buffered saline solution, potassium hydroxide aqueous solution, gastric mucin and lactic acid aqueous solution. The amount of gastric mucin added to the mucous component directly affects the final viscosity of the prepared AMF. A successful range of gastric mucin is usually between 38 to 50 grams. To prepare about 500 mL of the mucous component, add 460±10 mL of the previously prepared phosphate buffered saline solution and 7.5±0.5 mL of the 10% w/v potassium hydroxide aqueous solution to a 1000 mL heavy duty glass beaker. Place this beaker onto a stirring hot plate and while stirring, bring the temperature to 45° C.±5 C°. Weigh the pre-determined amount of gastric mucin (±0.50 g) and slowly sprinkle it, without clumping, into the previously prepared liquid that has been brought to 45° C. Cover the beaker and continue mixing. Over a period of 15 minutes bring the temperature of this mixture to above 50° C. but not to exceed 80° C. Continue heating with gentle stirring for 2.5 hours while maintaining this temperature range, then remove the beaker from the hot plate and cool to below 40° C. Next add 1.8±0.2 mL of the 10% v/v lactic acid aqueous solution and mix thoroughly. Autoclave the mucous component mixture at 121° C. for 15 minutes and allow 5 minutes for cool down. Remove the mixture of mucous component from the autoclave and stir until the temperature reaches 23° C.±1 C°.

Allow the temperature of the sheep blood and mucous component to come to 23° C.±1 C°. Using a 500 mL graduated cylinder, measure the volume of the entire batch of the mucous component and add it to a 1200 mL beaker. Add an equal volume of sheep blood to the beaker and mix thoroughly. Using the viscosity method previously described, ensure the viscosity of the AMF is between 7.15-8.65 cSt. If not the batch is disposed and another batch is made adjusting the mucous component as appropriate.

The qualified AMF should be refrigerated at 4° C. unless intended for immediate use. AMF may be stored in an air-tight container at 4° C. for up to 48 hours after preparation. Prior to testing, the AMF must be brought to 23° C.±1 C°. Any unused portion is discarded after testing is complete.

4. Rewet Test

Rewet is measured for an absorbent article loaded with Artificial Menstrual Fluid (AMF).

The fluid amounts left on a topsheet, i.e. rewet under pressure of 0.1 psi are measured after 6.0 ml and 12.0 ml AMF are dispensed. All measurements are performed in a laboratory maintained at 23° C.±2 C.° and 50%±2% relative humidity.

Test products are removed from all packaging using care not to press down or pull on the products while handling. No attempt is made to smooth out wrinkles. The test products are conditioned at 23° C.±2 C.° and 50%±2% relative humidity for at least 2 hours prior to testing.

Place the test product onto a flat, horizontal surface with the body side facing up and load a strikethrough plate on the center of the test product to apply a pressure of 0.25 psi on the test product.

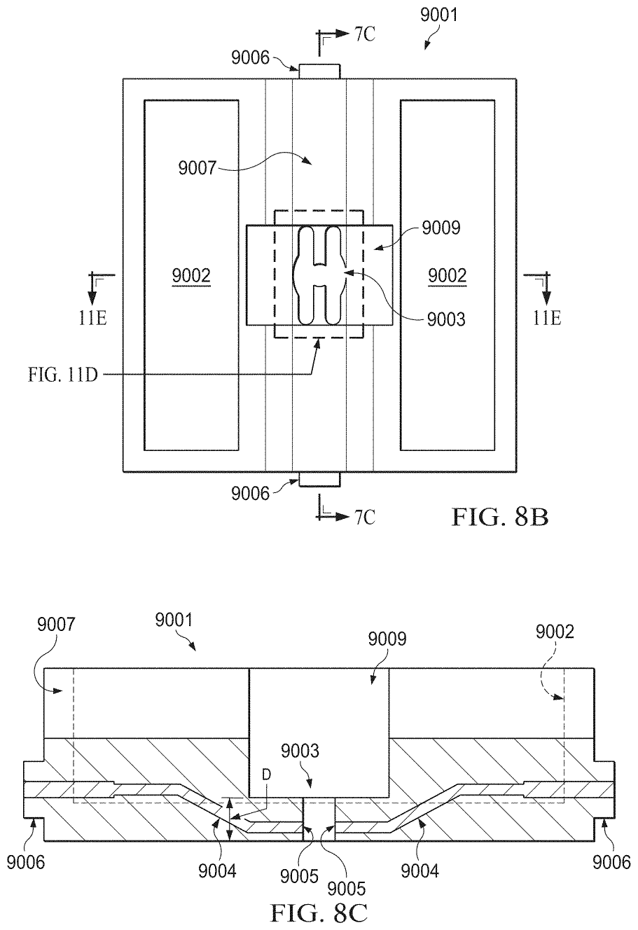

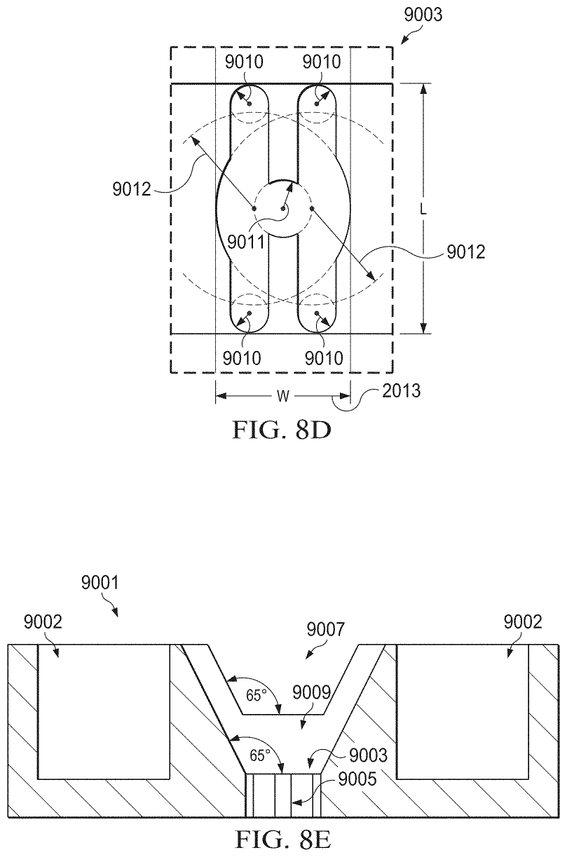

Referring to FIGS. 8A-8E, the strikethrough plate 9001 is constructed of Plexiglas with an overall dimension of 10.2 cm long by 10.2 cm wide by 3.2 cm tall. A longitudinal channel 9007 running the length of the plate is 13 mm deep and 28 mm wide at the top plane of the plate, with lateral walls that slope downward at 65° to a 15 mm wide base. A central test fluid well 9009 is 26 mm long, 24 mm deep and 38 mm wide at the top plane of the plate with lateral walls that slope downward at 65° to a 15 mm wide base. At the base of the test fluid well 9009, there is an “H” shaped test fluid reservoir 9003 open to the bottom of the plate for the fluid to be introduced onto the underlying article. The test fluid reservoir 9003 has an overall length (“L”) of 25 mm, width (“W”) of 15 mm, and depth (“D”) of 8 mm. The longitudinal legs of the reservoir are 4 mm wide and have rounded ends with a radius 9010 of 2 mm. The legs are 3.5 mm apart. The central strut has a radius 9011 of 3 mm and houses the opposing electrodes 9004 6 mm apart. The lateral sides of the reservoir bow outward at a radius 9012 of 14 mm bounded by the overall width, W, of 15 mm Two wells 9002 (80.5 mm long×24.5 mm wide×25 mm deep) located outboard of the lateral channel, are filled with lead shot to adjust the overall mass of the plate to provide a constraining pressure of 0.25 psi (17.6 gf/cm²) to the test area. Electrodes 9004 are embedded in the plate 9001, connecting the exterior banana jacks 9006 to the inside wall of the fluid reservoir 9003. A circuit interval timer is plugged into the jacks 9006 to the inside wall 9005 of the fluid reservoir 9003.

Use a pipette to carefully dispense 3.0 ml of AMF through the open hole of the strikethrough plate onto the center of the test articles within 2 seconds. Once the gush fluid is acquired, remove the plate and start the timer for 3 minutes. After removing the plate, quickly acquire an image of a topsheet of the test product using a color scanner HP Scanjet G4010 or equivalent, and clean the scanner surface after each scan. The image will be analyzed to measure an stain area on a topsheet under Stain Area Test below. At the end of 3 minutes, place 5 pieces of filter paper (a typical lab filter paper, for example, Ahlstrom #632 12.7 cm×12.7 cm filter papers) that are pre-weighed (termed as “dry weight”) are placed on top of an approximate center of an area stained with the fluid. Apply the required mass to generate 0.1 psi pressure on the top of the test product, and keep it under pressure for 5 seconds. Weigh the filter papers again (termed as “wet weight”). The difference between the wet weight and dry weight of the filter paper is the light pressure rewet at the added amount of fluid.

Repeat the step above till total 12.0 ml of fluid is dispensed on the test product. Report the rewet values to the nearest 0.001 gram for the gush level of 6.0 ml and 12.0 ml.

In like fashion, a total of three replicate samples are tested for each test product to be evaluated. Report the light pressure rewet as the arithmetic mean of the replicates to the nearest 0.001 gram.

5. Stain Area Test

The area of a stain visible on a topsheet of an absorbent article due to the fluid left on the topsheet is measured on topsheet images of test products acquired in Rewet Test above for the gush level of 6.0 ml and 12.0 ml.

Image analysis is performed using image analysis program such as Image J software (version 1.52p or above, National Institute of Health, USA) or equivalent. The image needs to be distance calibrated with an image of a ruler to give an image resolution, i.e. 7.95 pixels per mm.

Open a topsheet image in Image J. Set the scale according to the image resolution. Crop the image in the center area to make a minimum bounding rectangular selection around the total stain region visible across multiple pad layers. Convert the image type to 8 bit. Apply a Gaussian blur filter to smooth the image by a Gaussian function with a Sigma (radius) of 2. The filtered 8-bit grayscale image is then converted to a binary image using the “Minimum” thresholding method to find the boundary of the stain region on the topsheet (as a result of fluid left on the topsheet) against the lighter-colored stain region from the subsequent layers.

The area of the selected stain region on the topsheet is obtained and recorded as topsheet stain area to the nearest 0.01 cm². This entire procedure is repeated on three substantially similar replicate articles. The reported value is the average of the three individual recorded measurements for topsheet stain area to the nearest 0.01 cm².

### EXAMPLES

### Example 1. Nonwoven Substrate Preparation

Nonwovens 1-9: Carded air-through nonwovens made from 2 denier PET/PE core/sheath bicomponent fibers were produced according to Table 1 below were produced using a conventional process. In case of apertured nonwovens, obtained carded air-through nonwovens were put into a mechanical aperturing process comprising a pair of rolls to obtain apertured nonwoven substrates. For reloft, apertured nonwoven substrates were transferred to a reloft unit and relofted by providing the nonwoven substrate with hot air at a temperature about 80° C.˜about 110° C., or irradiating the nonwoven substrate with infrared radiation from a radiation source, to have a caliper in a first area is at least 1.2 times the initial caliper.

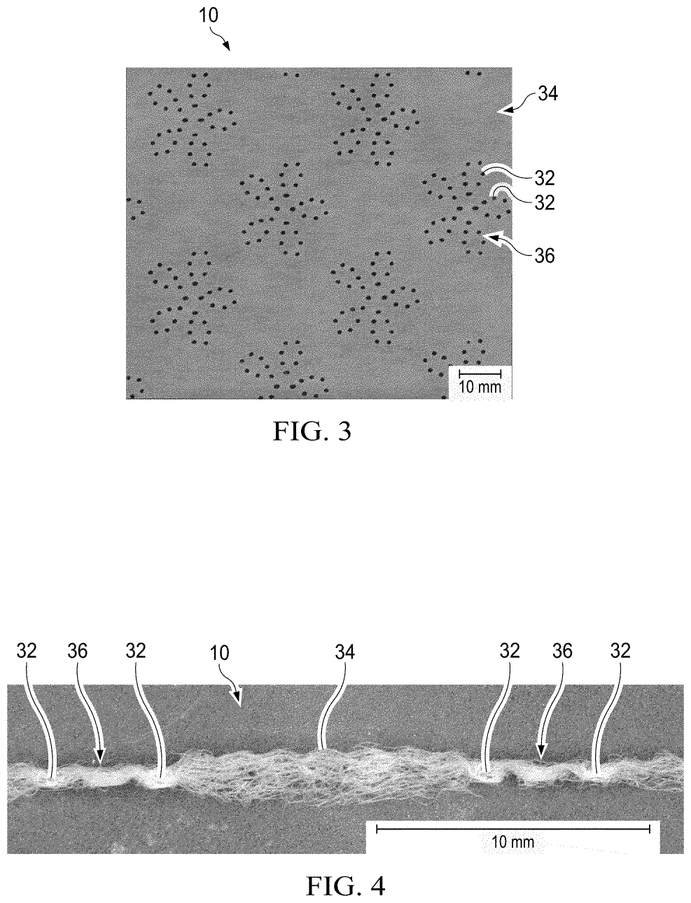

Nonwoven 10: A 50 gsm carded air-through nonwoven having a top layer nonwoven made from 2 denier PET/PE core/sheath bicopolymer fibers and a bottom layer nonwoven made from 2 denier PET/PE core/sheath bicomponent fibers and 6 denier PET fibers was produced. The nonwoven was put into a mechanical aperturing process comprising a pair of rolls to obtain an apertured nonwoven substrate having an aperture pattern shown in FIG. 3.

TABLE 1

| NW | NW 1 | NW 2 | NW 3 | NW 4 | NW 5 | NW 6 |
|---|---|---|---|---|---|---|
| Basis weight (gsm) | 24 | 24 | 24 | 24 | 33 | 33 |
| Aperture | No aperture | No aperture | FIG. 1 | FIG. 1 | No aperture | No aperture |
| Reloft | No reloft | Reloft | No reloft | Reloft | No reloft | Reloft |

| NW | NW 7 | NW 8 | NW 9 | NW 10 | NW 11 |
|---|---|---|---|---|---|
| Basis weight (gsm) | 33 | 33 | 24 | 50 | 24 |
| Aperture | FIG. 1 | FIG. 1 | FIG. 2 | FIG. 3 | No aperture |
| Reloft | No reloft | Reloft | Reloft | No reloft | Reloft |

### Example 2. Nonwoven Substrate Test

The obtained nonwoven substrates were evaluated as described below. Calipers of nonwovens were measured according to Caliper Test under TEST METHODS. Compression works of nonwoven substrates were measured according to Compression Property Test under the TEST METHODS. All results are indicated in Table 2 below.

TABLE 2

| Nonwoven | NW 1 | NW 2 | NW 3 | NW 4 | NW 5 | NW 6 | NW 7 | NW 8 |
|---|---|---|---|---|---|---|---|---|
| Compression | 190 | 1031 | 453 | 1167 | 146 | 1070 | 756 | 1346 |
| Work (gf × mm) | | | | | | | | |

| | | Nonwoven | | | | | | | | | | | |
|---|---|---|---|---|---|---|---|---|---|---|---|---|---|
| | | NW 3 | | NW 4 | | NW 7 | | NW 8 | | NW 9 | | NW 10 | |
| | | 1^{st} | 2^{nd} | 1^{st} | 2^{nd} | 1^{st} | 2^{nd} | 1^{st} | 2^{nd} | 1^{st} | 2^{nd} | 1^{st} | 2^{nd} |
| | | area | area | area | area | area | area | area | area | area | area | area | area |
| Caliper | at 0.1 psi | 771 | 636 | 1136 | 628 | 598 | 656 | 1193 | 713 | 749 | 582 | 1345 | 900 |
| (μm) | at 1.6 psi | 239 | 346 | 237 | 292 | 175 | 327 | 229 | 365 | 197 | 205 | 372 | 488 |

| Caliper ratio at 0.1 psi | 1.21 | | 1.81 | | 0.91 | | 1.67 | | 1.29 | | 1.49 | |
|---|---|---|---|---|---|---|---|---|---|---|---|---|
| Recovery caliper (μm) | 598 | 549 | 835 | 517 | 435 | 556 | 853 | 594 | 576 | 447 | 1041 | 773 |
| at 0.1 psi | | | | | | | | | | | | |
| Airy index (%) | 223 | | 379 | | 242 | | 421 | | 280 | | 262 | |
| Recovery airy index (%) | 150 | | 252 | | 149 | | 272 | | 192 | | 180 | |

### Example 3. Absorbent Articles

Sanitary napkins 1-4 as exemplary absorbent articles having topsheets made by nonwoven substrates according to Example 1 above were fabricated using a common secondary topsheet, an absorbent core and a backsheet. Sanitary napkins 5 and 6 as exemplary absorbent articles having topsheets made by nonwoven substrates according to Example 1 above were fabricated using a common secondary topsheet, an absorbent core and a backsheet. The secondary topsheet and absorbent core in Sanitary napkins 5 and 6 were different ones for Sanitary napkins 1-4.

Rewet and stain perception of sanitary napkin samples were measured according to Rewet Test and Stain Area Test under TEST METHODS above and indicated in Table 3. Sanitary napkins 1-4 were tested in the same batch, and Sanitary napkins 5 and 6 were tested in a separate batch.

TABLE 3

| | | Sanitary napkin | | | | | |
|---|---|---|---|---|---|---|---|
| | | 1 | 2 | 3 | 4 | 5 | 6 |
| Nonwoven | | NW 2 | NW 4 | NW 6 | NW 8 | NW 9 | NW 11 |
| Rewet | 6 ml | 0.026 | 0.012 | 0.035 | 0.022 | 0.04 | 0.03 |
| at 0.1 psi/g | 12 ml | 0.046 | 0.019 | 0.046 | 0.028 | 0.32 | 0.34 |
| Stain Area | 6 ml | 19.10 | 9.98 | 12.95 | 11.24 | 16.58 | 13.94 |
| (cm²) | 12 ml | 34.73 | 19.41 | 24.40 | 17.39 | 25.98 | 18.99 |

Sanitary napkins 2 and 4 having topsheets made by nonwoven substrate according to the present invention have significantly improved rewet in comparison with Sanitary napkins 1 and 3. In addition, Sanitary napkins 2 and 4 has much lower strain perception than Sanitary napkins 1 and 3.

Sanitary napkin 5 with a nonwoven topsheet having non-clustered apertures does not show improvements in either rewet or in strain perception in comparison to Sanitary napkin 6.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An apertured nonwoven substrate comprising:
a first area having a first caliper,
a second area comprising a plurality of apertures, the second area having a second caliper,
wherein a ratio of the first caliper to the second caliper is at least about 1.35 as measured according to Caliper Test, and
wherein the first area has an airy index no less than about 270%, as measured according to Caliper Test.

2. The apertured nonwoven substrate according to claim 1, wherein the first area has a recovery airy index no less than about 160%, as measured according to Caliper Test.

3. The apertured nonwoven substrate according to claim 1, wherein the nonwoven substrate is a relofted nonwoven.

4. The apertured nonwoven substrate according to claim 1, wherein the second area comprises at least one aperture having at least three adjacent apertures, wherein the one aperture and each of the at least three adjacent apertures has an edge to edge space no greater than about 3 mm.

5. The apertured nonwoven substrate according to claim 1, wherein the nonwoven substrate has a compression work no less than about 700 gf×mm, as measured according to Compression Property Test.

6. The apertured nonwoven substrate according to claim 1, wherein the nonwoven substrate has a basis weight in the range of about 15 gsm to about 75 gsm.

7. A relofted apertured nonwoven substrate comprising:
a first area comprising no aperture,
a second area comprising at least one aperture having at least three adjacent apertures, wherein an edge to edge space between the one aperture and each of the at least three adjacent apertures is no greater than about 3 mm.

8. The relofted apertured nonwoven substrate according to claim 7, wherein the first area has an airy index no less than about 270% as measured according to Caliper Test.

9. The relofted apertured nonwoven substrate according to claim 7, wherein the nonwoven substrate has a basis weight in the range of about 15 gsm to about 75 gsm.

10. The relofted apertured nonwoven substrate according to claim 7, wherein the second area comprises highly heat-fused fibers.

11. The relofted apertured nonwoven substrate according to claim 7, wherein fibers in peripheries of the plurality of apertures are more compressed and heat-fused than the fibers in the first area.

12. An absorbent article comprising a liquid pervious topsheet, a liquid impervious backsheet, an absorbent structure disposed between the topsheet and the backsheet, and the relofted apertured nonwoven substrate according to claim 7.

13. The absorbent article of claim 12, wherein the topsheet comprises the relofted apertured nonwoven substrate.

14. The absorbent article of claim 12, wherein the backsheet comprises the relofted apertured nonwoven substrate.

15. A method for manufacturing an apertured nonwoven substrate, comprising the steps of:
forming a plurality of apertures on a nonwoven web, and
applying energy to the nonwoven web to increase bulkiness of the nonwoven web.

16. The method according to claim 15, wherein the apertured nonwoven substrate comprises:
a first area having a first caliper,
a second area comprising a plurality of apertures, the second area having a second caliper,
wherein the second area comprises at least one aperture having at least three adjacent apertures, wherein an edge to edge space between the one aperture and each of the at least three adjacent apertures is no greater than about 3 mm.

17. The method according to claim 15, wherein a ratio of the first caliper to the second caliper is at least about 1.35, as measured according to Caliper Test.

18. The method according to claim 15, wherein the first area has an airy index no less than about 270%, as measured according to Caliper Test.

19. The method of claim 15, wherein the apertured nonwoven substrate has a basis weight in the range of about 15 gsm to about 75 gsm.

20. The method according to claim 15, wherein the apertured nonwoven substrate has a compression work no less than about 700 gf×mm, as measured according to Compression Property Test.
